(19) United States
(12) Patent Application Publication
(10) Pub. No.: US 2026/0325115 A1
(43) Pub. Date: Oct. 22, 2026

(54) ORAL SPECIMEN COLLECTION APPARATUS

(71) Applicant: Board of Regents of the University of Nebraska, Lincoln, NE (US)

(72) Inventors: Thang Nguyen, Elkhorn, NE (US); Michael Wadman, Omaha, NE (US); Jared Marx, Omaha, NE (US); Abraham Campos, Papillion, NE (US); Aaron Barksdale, Elkhorn, NE (US)

(21) Appl. No.: 19/122,954

(22) PCT Filed: Oct. 27, 2023
(86) PCT No.: PCT/US2023/078048

§ 371 Date: Apr. 21, 2025

Related U.S. Application Data
(60) Provisional application No. 63/381,250, filed on Oct. 27, 2022.

(51) Int. Cl. A61B 10/00

(52) U.S. Cl. CPC A61B 10/0051

(57) ABSTRACT

An oral specimen collection apparatus comprises a deformable container including one or more collection ports, wherein a negative pressure is produced within the deformable container when the deformable container is compressed within an oral cavity, and wherein the negative pressure causes a specimen to be suctioned into the deformable container through the one or more collection ports as the deformable container decompresses.

### CROSS-REFERENCE TO RELATED APPLICATIONS

The present application claims priority under 35 U.S.C. § 119(e) to U.S. Provisional Application Ser. No. 63/381,250, filed Oct. 27, 2022, and titled “ORAL SPECIMEN COLLECTION APPARATUS,” which is incorporated herein by reference in its entirety.

### TECHNICAL FIELD

The present invention relates to apparatus, systems, and methods for collection of oral specimens.

### BACKGROUND

Saliva testing or Salivaomics is a diagnostic technique that involves laboratory analysis of saliva to identify markers of endocrine, immunologic, inflammatory, infectious, and other types of conditions. Saliva is a useful biological fluid for assaying steroid hormones such as cortisol, genetic material like RNA, proteins such as enzymes and antibodies, and a variety of other substances, including natural metabolites, including saliva nitrite, a biomarker for nitric oxide status. Saliva testing is used to screen for or diagnose numerous conditions and disease states, including but not limited to, COVID-19, malaria, Cushing's disease, anovulation, HIV, cancer, parasites, hypogonadism, and allergies. Salivary testing has even been used by the U.S. government to assess circadian rhythm shifts in astronauts before flight and to evaluate hormonal profiles of soldiers undergoing military survival training.

The typical process of collecting specimens for saliva testing requires swabbing of the oropharyngeal cavity with a cotton swab. This process is invasive and uncomfortable for the patient being swabbed. The procedure can trigger the patient's gag reflex leading to coughing, sneezing, or vomiting; placing the healthcare professional at risk for infection. Resistance or hesitation by the patient can result in suboptimal specimen collection resulting in a false negative result and improper treatment. In some cases, patients may even refuse testing due to the discomfort associated with the specimen collection procedure.

To improve saliva testing procedures and patient compliance, there is a need for less invasive and more comfortable oral specimen collection apparatus and techniques. There is also need for diagnostic tests that can be self-administered to prevent/reduce the risk of contagion.

### SUMMARY

An oral specimen collection (OSC) apparatus is disclosed. In embodiments, the OSC apparatus comprises a deformable container including one or more collection ports, wherein a negative pressure is produced within the deformable container when the deformable container is compressed within an oral cavity, and wherein the negative pressure causes a specimen to be suctioned into the deformable container through the one or more collection ports as the deformable container decompresses.

The OSC apparatus presented herein may offer a number of advantages including, but not limited to, improved patient comfort (e.g., reduced/no pain), fast sampling, accurate results, higher patient acceptance/compliance, and/or minimized infection risk for healthcare professionals.

This Summary is provided solely as an introduction to subject matter that is fully described in the Detailed Description and Drawings. The Summary should not be considered to describe essential features nor be used to determine the scope of the Claims. Moreover, it is to be understood that both the foregoing Summary and the following Detailed Description are example and explanatory only and are not necessarily restrictive of the subject matter claimed.

### BRIEF DESCRIPTION OF THE DRAWINGS

The Detailed Description is provided with reference to the accompanying Drawings. The use of the same reference numbers in different instances in the Detailed Description and the Drawings may indicate similar or identical items. The Drawings are not necessarily to scale, and any disclosed processes may be performed in an arbitrary order, unless a certain order of steps/operations is inherent or specified in the Detailed Description or in the Claims.

FIG. 1A illustrates a process by which an oral specimen collection (OSC) apparatus is placed within an oral cavity to collect an oral specimen, in accordance with one or more embodiments of this disclosure.

FIG. 1B illustrates a process by which an OSC apparatus is compressed within an oral cavity to produce a negative pressure within the OSC apparatus that then acts on fluid within the oral cavity to suction a specimen into the OSC apparatus as the OSC apparatus decompresses, in accordance with one or more embodiments of this disclosure.

FIG. 2 is a perspective view of an OSC apparatus comprising a bullet shaped deformable container with an elongated handle for positioning and stabilizing the deformable container within an oral cavity, in accordance with one or more embodiments of this disclosure.

FIG. 3 is a perspective view of an OSC apparatus comprising a deformable container with a substantially rectangular compression zone cross-section and an elongated handle for positioning and stabilizing the deformable container within an oral cavity, in accordance with one or more embodiments of this disclosure.

FIG. 4 is a perspective view of an OSC apparatus comprising a disk shaped deformable container with an elongated handle for positioning and stabilizing the deformable container within an oral cavity, in accordance with one or more embodiments of this disclosure.

FIG. 5 is a perspective view of an OSC apparatus comprising a disk shaped deformable container with a loop handle for positioning and stabilizing the deformable container within an oral cavity, in accordance with one or more embodiments of this disclosure.

FIG. 6 is a perspective view of an OSC apparatus comprising a deformable container with a substantially rectangular compression zone cross-section, wherein the OSC apparatus includes a dropper/output port that is separate from its collection ports, in accordance with one or more embodiments of this disclosure.

FIG. 7A illustrates a test kit that includes an OSC apparatus, in accordance with one or more embodiments of this disclosure.

FIG. 7B further illustrates the test kit of FIG. 7A, wherein a collected specimen and a reagent are added to a test tube, in accordance with one or more embodiments of this disclosure.

FIG. 7C further illustrates the test kit of FIG. 7A, wherein a test strip is placed within the test tube after the collected specimen and reagent have been added to the test tube, in accordance with one or more embodiments of this disclosure.

FIG. 8 is a perspective view of a modular system that includes a reagent container and a testing container that are connected to or configured to interface with an OSC apparatus, in accordance with one or more embodiments of this disclosure.

FIG. 9A is a front view of an OSC apparatus comprising a deformable container with a substantially circular or elliptical compression zone cross-section, wherein the OSC apparatus includes a dropper/output port that is separate from its collection ports, in accordance with one or more embodiments of this disclosure.

FIG. 9B is a cross-sectional perspective view of the OSC apparatus of FIG. 9A, in accordance with one or more embodiments of this disclosure.

FIG. 9C is a perspective view of the OSC apparatus of FIG. 9A, wherein the OSC apparatus includes recoil structures that assist with shape recovery of its compression zone, in accordance with one or more embodiments of this disclosure.

FIG. 10 is a perspective view of the OSC apparatus of FIG. 9A, wherein the OSC apparatus includes removable end caps configured to seal its collection ports and dropper/output port, in accordance with one or more embodiments of this disclosure.

FIG. 11A is a perspective view of a system that includes the OSC apparatus of FIG. 9A and a docking plate for the OSC apparatus, wherein the docking plate includes plugs configured to seal the collection ports and dropper/output port of the OSC apparatus, in accordance with one or more embodiments of this disclosure.

FIG. 11B is another perspective view of the system of FIG. 11A, wherein the system further includes a reagent container/cartridge and a testing container/cartridge that are connected to or configured to interface with the OSC apparatus via the docking plate, in accordance with one or more embodiments of this disclosure.

FIG. 12A is a perspective view of an OSC apparatus comprising a deformable container with a substantially circular or elliptical compression zone cross-section, wherein the OSC apparatus includes a dropper/output port that is separate from its collection ports, and wherein the OSC apparatus further includes vertical grooves on an outer surface of the compression zone, wherein the vertical grooves are configured to direct saliva flowing across the outer surface of the compression zone into a drain ports that feed into the OSC apparatus, in accordance with one or more embodiments of this disclosure.

FIG. 12B is a top view of the OSC apparatus of FIG. 12A, in accordance with one or more embodiments of this disclosure.

FIG. 12C is a cross-sectional side view of the OSC apparatus of FIG. 12A, in accordance with one or more embodiments of this disclosure.

FIG. 13A is a perspective view of an OSC apparatus comprising a deformable container with a substantially circular or elliptical compression zone cross-section, wherein the OSC apparatus includes a dropper/output port that is separate from its collection ports, and wherein the OSC apparatus further includes helical grooves on an outer surface of the compression zone, wherein the helical grooves are configured to direct saliva flowing across the outer surface of the compression zone into a drain ports that feed into the OSC apparatus, in accordance with one or more embodiments of this disclosure.

FIG. 13B is a side view of the OSC of FIG. 13A, in accordance with one or more embodiments of this disclosure.

FIG. 13C is a top view of the OSC of FIG. 13A, in accordance with one or more embodiments of this disclosure.

FIG. 14A is a partially exploded perspective view of a system including an OSC attachment and a base structure, wherein the base structure is configured to be coupled to the OSC attachment in order to collect an oral specimen within the base structure via the OSC attachment, and wherein the base structure is further configured to be coupled to a sample tube after collecting the oral specimen in order to transfer the oral specimen to the sample tube, in accordance with one or more embodiments of this disclosure.

FIG. 14B is a perspective view of the system of FIG. 14A, in accordance with one or more embodiments of this disclosure.

FIG. 14C is a cross-sectional perspective view of the system of FIG. 14A, in accordance with one or more embodiments of this disclosure.

FIG. 14D is a cross-sectional perspective side view of the system of FIG. 14A, in accordance with one or more embodiments of this disclosure.

FIG. 14E is a cross-sectional side view of the system of FIG. 14A after the OSC attachment has been removed from the base structure and the base structure has been coupled to a sample tube, in accordance with one or more embodiments of this disclosure.

FIG. 15A is a perspective view of an OSC attachment, in accordance with one or more embodiments of this disclosure.

FIG. 15B is a perspective end view of the OSC attachment of FIG. 15A, in accordance with one or more embodiments of this disclosure.

FIG. 15C is a partially exploded cross-sectional side view of a system including the OSC attachment of FIG. 15A and a base structure, wherein the base structure is configured to be coupled to the OSC attachment in order to collect an oral specimen within the base structure via the OSC attachment, and wherein the base structure is further configured to be coupled to a sample tube after collecting the oral specimen in order to transfer the oral specimen to the sample tube, in accordance with one or more embodiments of this disclosure.

FIG. 15D is a side view of the system of FIG. 15C, in accordance with one or more embodiments of this disclosure.

FIG. 15E is a cross-sectional side view of the system of FIG. 15C, in accordance with one or more embodiments of this disclosure.

### DETAILED DESCRIPTION

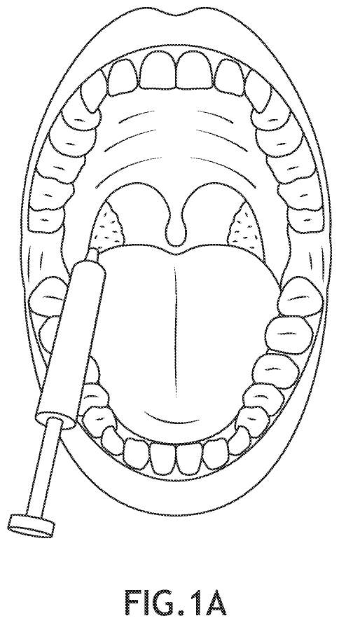

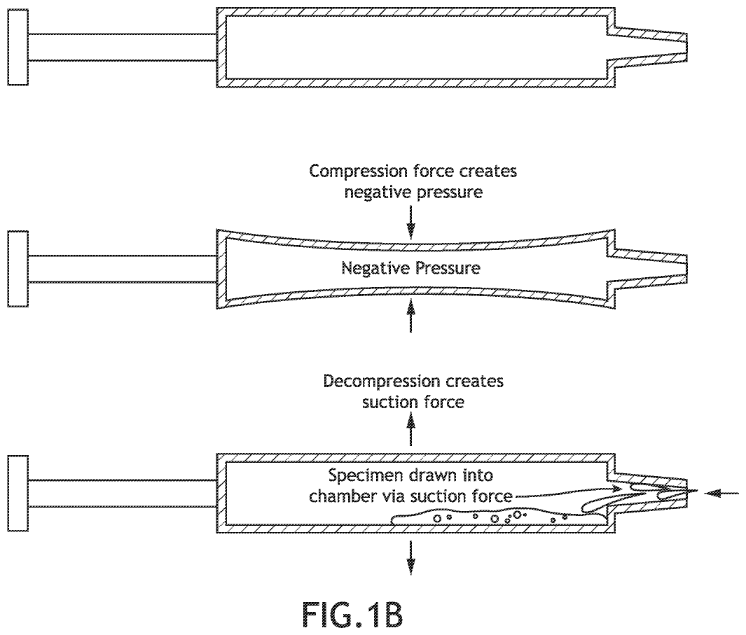

This disclosure presents an oral specimen collection (OSC) apparatus that generally comprises a hollow deformable container, preferably made of soft plastic material (although other materials may be appropriate), with one or more small openings (collection ports) at the distal end. In an example use scenario, a patient is asked to insert the deformable container into their mouth between their upper and lower molars/gums, with distal end pointed toward their oropharyngeal cavity (see, for example, FIG. 1A). The patient is then asked to chew and/or suck on the deformable container. As illustrated in FIG. 1B, compression and decompression of the deformable container creates a suction force that draws specimen (e.g., saliva, epithelial cells, etc.) from the oropharyngeal cavity into the deformable container through one or more collection ports. When the OSC apparatus is compressed within an oral cavity, this action produces a negative pressure that then acts on fluid within the oral cavity to suction a specimen (i.e., a portion of the fluid and its contents) into the OSC apparatus as the OSC apparatus decompresses.

The OSC apparatus does not necessarily have to be inserted between upper and lower molars/gums. Any type of compressive action (e.g., bite force, mastication, any applied tongue/mouth pressure, or other compressive force) within the oral cavity may suffice to draw fluid (e.g., a saliva specimen) into the OSC apparatus via one or more collection ports.

Furthermore, the patient's interaction with the OSC apparatus may promote salivation. For example, it has been observed that the act of mastication (biting/chewing) plays a key role in the promotion of salivation. In some embodiments, at least a portion of the OSC apparatus's external structure may be coated with a flavored (e.g., sweet and/or sour) powder, syrup, or any other flavored granular/fluid coating to promote user acceptance (especially in children) and to activate salivation.

Some embodiments of the OSC apparatus include a passive drainage system to take advantage of the patient's increased salivation while using the device. Passive drainage of saliva, as observed with the OSC apparatus, minimizes the introduction of specimen artifacts such as bubbles, mucus, contaminants (e.g., food, and so forth). In clinical trials, spit saliva seemed to have higher concentrations of bubbles and was more viscous (possibly from mucus). These artifacts can hinder testing.

In preliminary testing, the OSC apparatus was successful in collecting an oropharyngeal specimen that tested positive for a strep throat infection while yielding a stronger lateral flow response than the traditional swab technique. Test subjects also reported higher acceptance of the OSC apparatus, reporting no pain or discomfort with the procedure. The OSC apparatus can be used in any age group. Application in the pediatric population is particularly appealing as children tend to be most resistive to the swab method.

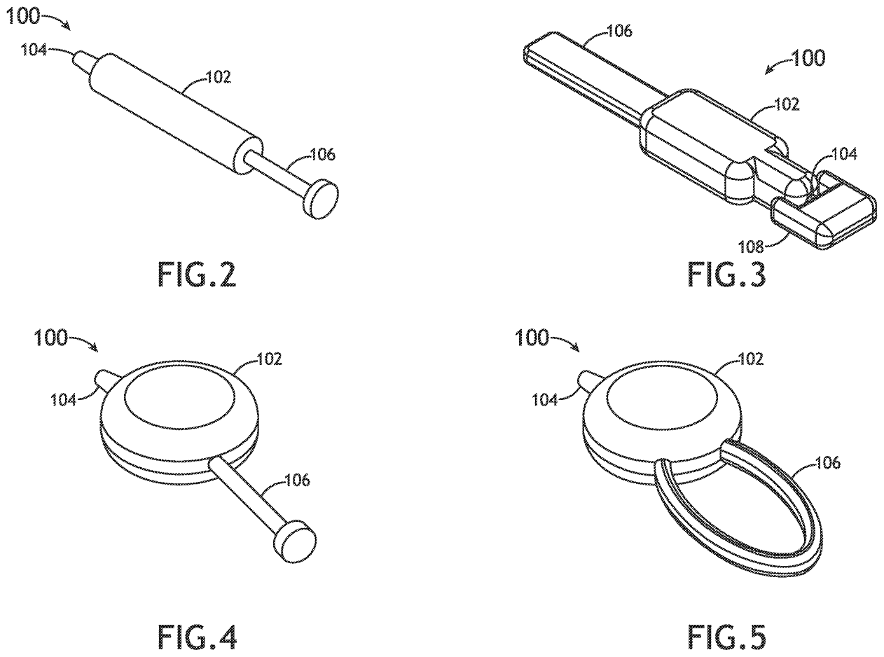

FIGS. 2 through 15E illustrate various embodiments of the OSC apparatus. Those skilled in the art will appreciate that the embodiments illustrated in FIGS. 2 through 15E are specific examples that can be modified by changing dimensions or adding/removing non-essential components or fillers. Furthermore, two or more embodiments (or portions thereof) can be combined to achieve an additional embodiment that is not necessarily shown or described with reference to one drawing. As such, the drawings should not be construed as restrictive of any particular embodiment and are intended instead as visual aids to help describe configurations of certain components that may apply to multiple embodiments of the OSC apparatus.

FIGS. 2 through 5 illustrate different designs for an OSC apparatus 100 that uses the same port or set of ports for specimen collection and output. In the embodiments illustrated in FIGS. 2 through 5, the OSC apparatus 100 includes a deformable container 102 with one or more collection ports 104 at its distal end, wherein a negative pressure is produced within the deformable container 102 when the deformable container 102 is compressed within an oral cavity, and wherein the negative pressure causes a specimen to be suctioned into the deformable container 102 through the one or more collection ports 104 as the deformable container 102 decompresses.

The OSC apparatus 100 also includes one or more output ports for transferring the specimen from the deformable container to a sample tube (e.g., a test tube, vial, collection tube, centrifuge tube, or any other type of container) for testing, transporting, and/or storing the specimen. In the embodiments illustrated in FIGS. 2 through 5, the same opening or set of openings that define the collection port 104 is used as an output port. For example, after collecting an oral specimen through the collection port 104, the OSC apparatus 100 may be held with its distal end pointed into a sample tube and squeezed to transfer its contents (i.e., the collected specimen) into the sample tube. In some embodiments, to facilitate easier transfer from the OSC apparatus to the sample tube for testing or any other purpose, the collection/output port 104 may be located at the end of a dropper tip.

In some embodiments, the OSC apparatus 100 may include grip portion 106, such as a handle (e.g., see FIGS. 2-4) and/or loop (e.g., see FIG. 5), that extends from a proximal end of the deformable container 102 to secure the device while in use and to prevent aspiration/swallowing of the device. As shown in FIG. 3, some embodiments of the OSC apparatus 100 may also include a removable end cap 108 that is configured to plug or otherwise seal the one or more collection/output ports 104 at the distal end of the OSC apparatus 100. For example, the end cap 108 may be attached to the distal end of the OSC apparatus 100 to seal its contents (i.e., a collected specimen) after the OSC apparatus 100 has been chewed and/or sucked on by the patient for a selected amount of time.

The OSC apparatus 100 may be manufactured in a variety of different shapes and sizes to accommodate different users (e.g., adults, children, etc.) and depending on whether the OSC apparatus 100 is configured to be self-administered or administered by a healthcare provider. In the latter situation, the OSC apparatus 100 may have a longer grip portion 106 that can be manipulated with less chance of contact with the patient's fluids. Alternative embodiments of the OSC apparatus 100 are illustrated in FIGS. 2 through 5, wherein the deformable container 102 is substantially bullet/barrel/cylindrically shaped (FIG. 2), polyhedral (FIG. 3), or disk shaped (FIGS. 4 and 5) with a handle/loop grip portion 106 for stabilizing the deformable container 102 within the oral cavity. Although certain geometries are provided as examples, the deformable container 102 can be any shape that sufficiently restores to its original shape upon being compressed and decompressed.

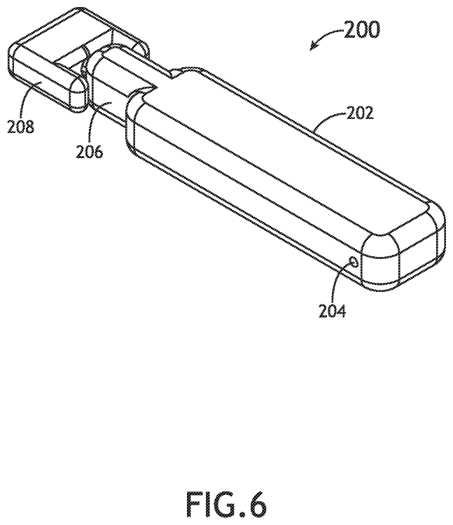

FIG. 6 illustrates another embodiment of an OSC apparatus 200, wherein the OSC apparatus 200 has separate collection and output ports disposed at opposite ends of the device. In the embodiment illustrated in FIG. 6, the OSC apparatus 200 includes a deformable container 202 with one or more collection ports 204 near its distal end, wherein a negative pressure is produced within the deformable container 202 when the deformable container 202 is compressed within an oral cavity, and wherein the negative pressure causes a specimen to be suctioned into the deformable container 202 through the one or more collection ports 204 as the deformable container 202 decompresses. In some embodiments, collection ports 204 are disposed on side surfaces or edges of the deformable container 202 (e.g., one on each side/edge as shown in FIG. 6) as this configuration may be less prone to occlusion of the collection ports 204.

The OSC apparatus 200 also includes one or more output ports for transferring the specimen from the deformable container 202 to a sample tube (e.g., a test tube, vial, collection tube, centrifuge tube, or any other type of container) for testing, transporting, and/or storing the specimen. In the embodiment illustrated in FIG. 6, the output port is at the end of a dropper tip 206 at an end of the deformable container 202 that is opposite from the collection ports 204. For example, after collecting an oral specimen through the collection ports 204, the OSC apparatus 200 may be held with its dropper tip 206 pointed into a sample tube and squeezed to transfer its contents (i.e., the collected specimen) into the sample tube.

In some embodiments, the OSC apparatus 200 may also include a removable end cap 208 that is configured to plug or otherwise seal the one or more output ports at the dropper tip 206 of the OSC apparatus 200. For example, the end cap 208 may be used to seal the output port in order to maintain pressure within the deformable container 202. The end cap 208 can then be removed when it is time to transfer contents of the deformable container 202 into the sample tube.

The OSC apparatus 200 may be manufactured in a variety of different shapes and sizes to accommodate different users (e.g., adults, children, etc.) and depending on whether the OSC apparatus 200 is configured to be self-administered or administered by a healthcare provider. In the embodiment illustrated in FIG. 6, the deformable container 202 is polyhedral (e.g., rectangular prism shaped) with a substantially polygonal (e.g., rectangular) cross-section. Although this geometry is provided as an example, the deformable container 102 can be any shape that sufficiently restores to its original shape upon being compressed and decompressed.

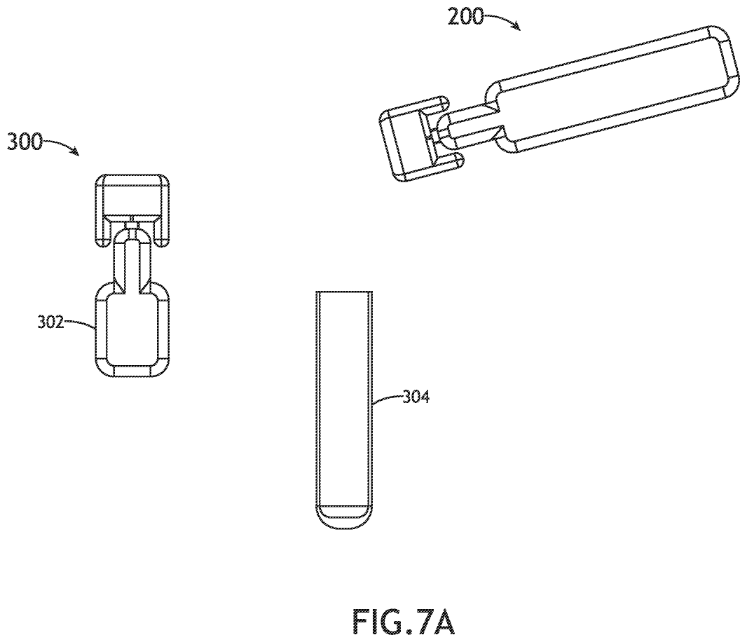

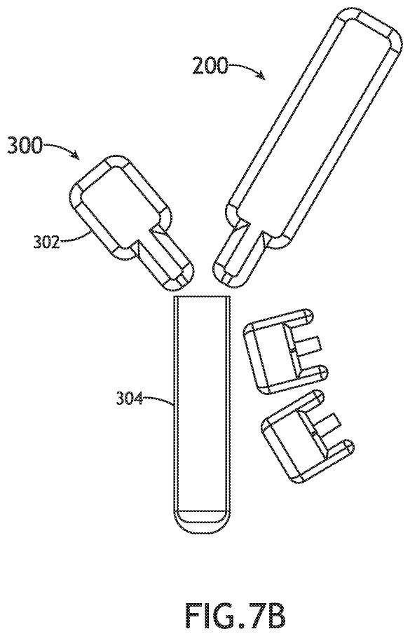

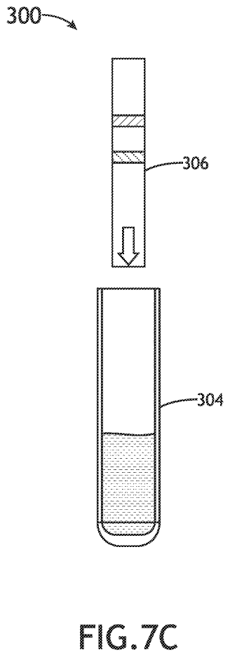

Referring now to FIGS. 7A through 7C, the OSC apparatus 200 may be part of a test kit 300, further including one or more reagent containers 302 (e.g., a reagent dropper), and a testing container 304 (e.g., a test tube or any other sample tube) configured to receive at least a portion of the specimen collected by the OSC apparatus 200 and one or more reagents from the one or more reagent containers 302. For example, the OSC apparatus 200 and the reagent container 302 can be used to drop a selected amount of the specimen and reagent into the testing container 304 (see FIG. 7B). As shown in FIG. 7C, the test kit 300 may further include a test strip 306 disposed within or configured to be disposed within the testing container 302 to perform a diagnostic test.

Although the test kit 300 of FIGS. 7A through 7C is described with reference to the OSC apparatus 200 illustrated in FIG. 6, the test kit 300 may be used with any other embodiment of the OSC apparatus described herein. For example, the test kit 300 can be used with OSC apparatus 100, OSC apparatus 500, OSC apparatus 700, OSC apparatus 800, or OSC apparatus 900 in place of OSC apparatus 200.

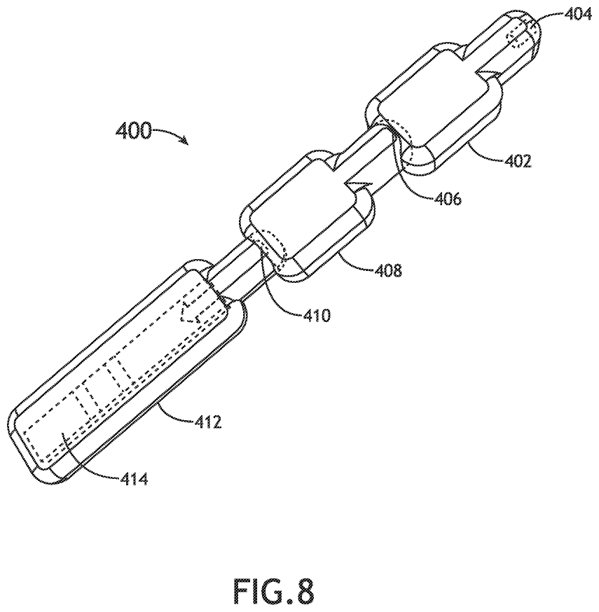

FIG. 8 is a perspective view of a modular system 400 that includes an OSC apparatus 402 having a collection port 404 at its distal end, wherein the modular system 400 further includes a reagent container 408 and a testing container 412 that are integrated within a common structure, coupled together, or configured to be coupled together. In embodiments, the modular containers may be separated by breakable seals 406 and 410 that, when broken, allow the specimen to flow from the OSC apparatus 402 to the one or more reagent containers 408 and then to the testing container 412. The fluid paths (e.g., openings) between the containers may be configured to provide a selected flow rate so that the specimen and reagent fluid mixture reaches the testing container 412 (optionally containing a test strip 414) at an appropriate flow rate and with an appropriate specimen concentration in the fluid mixture. Furthermore, to allow the reagent sufficient time to interact with the specimen, the seal 406 separating the OSC apparatus 402 and the reagent container 408 may be configured to be broken a selected period of time before breaking of the seal 410 between the reagent container 408 and the testing container 412.

As previously discussed, embodiments of the OSC apparatus may include collection ports located on edge or side portions of the distal end of the deformable container to prevent occlusion of the collection ports. For example, in the embodiment illustrated in FIG. 6, collection ports 204 are located on sides/edges of the deformable container 202 near its distal end, including at least a first collection port on a first side of the deformable container 202 near the distal end of the deformable container 202 and a second collection port on a second side of the deformable container 202 near the distal end of the deformable container 202.

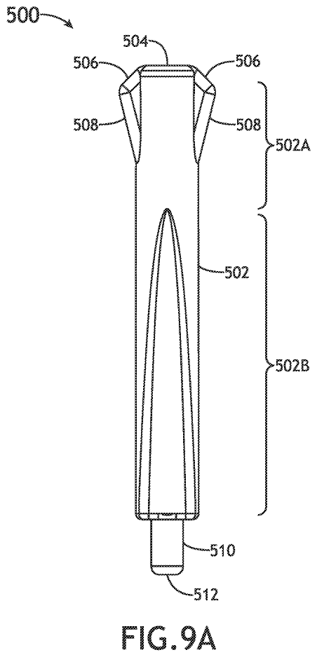

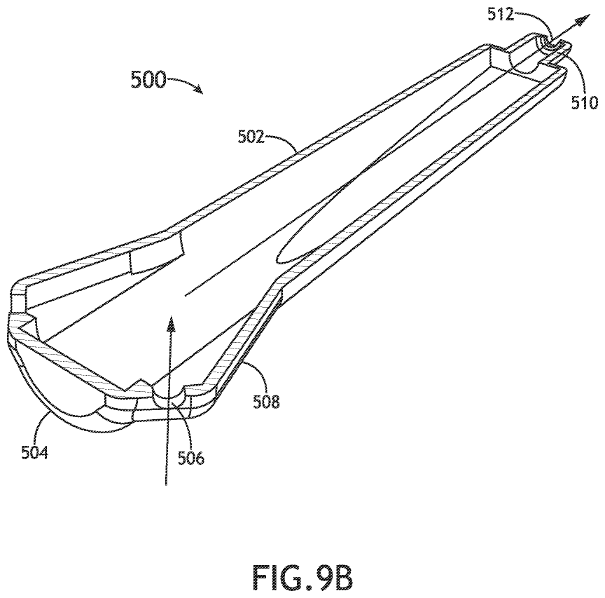

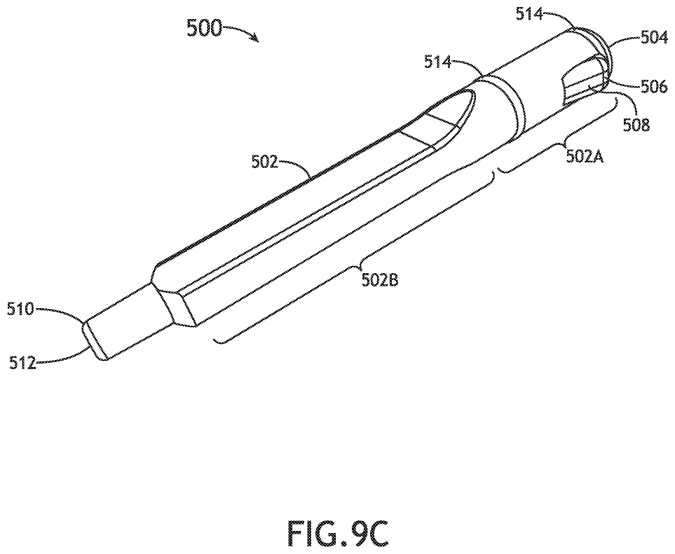

FIGS. 9A through 9C illustrate a more preferable embodiment, wherein an OSC apparatus 500 includes a deformable container 502 with first and second collection ports 506 formed on angled surfaces 508 (flared out portions) extending from first and second sides of the deformable container 502, respectively, near its distal end 504. As shown in FIG. 9A, the deformable container 502 comprises an elongated body with a compression zone 502A configured to be compressed within the oral cavity. The remaining portion 502B of the deformable container may specifically include a handle or may have a long enough body to be used as a handle to stabilize the compression zone portion 502A of the deformable container 502 within the oral cavity.

In some embodiments, the compression zone 502A has a substantially polygonal cross-section, such as the rectangular cross-section of the embodiment illustrated in FIG. 6. However, it has been found that a compression zone 502A with a circular or elliptical cross-section is better at restoring to its original shape upon being decompressed and can therefore provide better suction. Accordingly, FIGS. 9A through 9C illustrate a more preferable embodiment where the compression zone 502A has a substantially circular cross-section. Other embodiments may include a compression zone with an elliptical, semi-circular, or semi-elliptical shaped cross-section, or any other geometry that sufficiently restores to its original shape upon being compressed and decompressed.

As shown in FIG. 9C, the deformable container 502 may also be reinforced by one or more recoil structures 514 (e.g., recoil rings) to assist in returning the deformable container 502 to its original shape after the deformable container 502 is decompressed. The recoil structures 514 may be formed or embedded within the deformable container 502. For example, the recoil structures 514 (rings) illustrated in FIG. 9C comprise additional layers or thicker portions of plastic at selected positions in the deformable container 502. In particular, the recoil structures 514 may be located in the compression zone 502A to help improve shape restoration of the compression zone 502A after it has been chewed or otherwise compressed within a user's oral cavity.

Similar to the OSC apparatus 200 of FIG. 6, the OSC apparatus 500 of FIGS. 9A through 9C includes a deformable container 502 with one or more collection ports 506 near its distal end 504, wherein a negative pressure is produced within the deformable container 502 when the deformable container 502 (i.e., the compression zone 502A) is compressed within an oral cavity, and wherein the negative pressure causes a specimen to be suctioned into the deformable container 502 through the one or more collection ports 506 as the deformable container 502 decompresses. In some embodiments, collection ports 506 are disposed on angled surfaces 508 (flared out portions) extending from first and second sides of the deformable container 502, respectively, as this configuration may be less prone to occlusion of the collection ports 506.

The OSC apparatus 500 also includes one or more output ports 512 for transferring the specimen from the deformable container 502 to a sample tube (e.g., a test tube, vial, collection tube, centrifuge tube, or any other type of container) for testing, transporting, and/or storing the specimen. In the embodiments illustrated in FIGS. 9A through 9C, the output port 512 is at the end of a dropper tip 510 at an end of the deformable container 502 that is opposite from the collection ports 506. For example, after collecting an oral specimen through the collection ports 506, the OSC apparatus 500 may be held with its dropper tip 510 pointed into a sample tube and squeezed to transfer its contents (i.e., the collected specimen) into the sample tube.

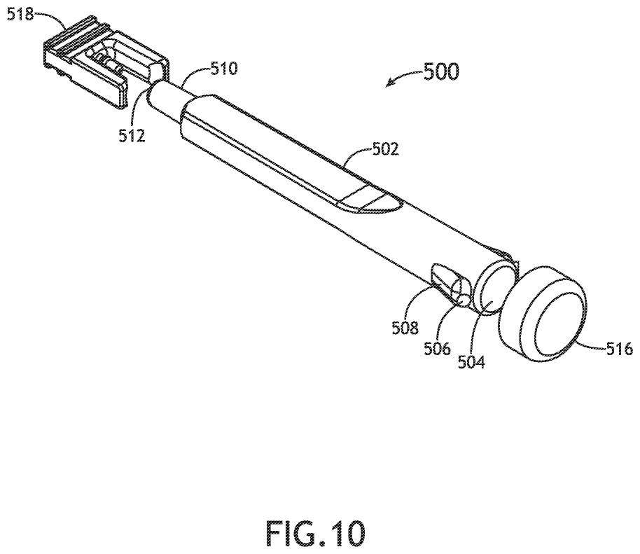

As shown in FIG. 10, in some embodiments, the OSC apparatus 500 may also include a removable end cap 516 that is configured to plug or otherwise seal the one or more collection ports 506 at the distal end 504 of the OSC apparatus 500 and/or a removable end cap 518 that is configured to plug or otherwise seal the one or more output ports 512 at the dropper tip 510 of the OSC apparatus 500. For example, end cap 518 may be used to seal the output port 512 in order to maintain pressure within the deformable container 502. The end cap 518 can then be removed when it is time to transfer contents of the deformable container 502 into the sample tube. Meanwhile, end cap 516 can be used to seal the collection ports 506 of the OSC apparatus 500 after a specimen has been collected to prevent the specimen from leaking back out of the deformable container 502 through the collection ports 506.

The removable end caps 516 and 518 can be used to seal collection and output ports and of the OSC apparatus 500, respectively, before and/or after specimen collection. In embodiments, the removable end caps 516 and 518 (or any other end cap disclosed herein with regard to this or another embodiment) may comprise plugs, pressure/friction fit caps, threaded caps, or any other type of cap structure or seal.

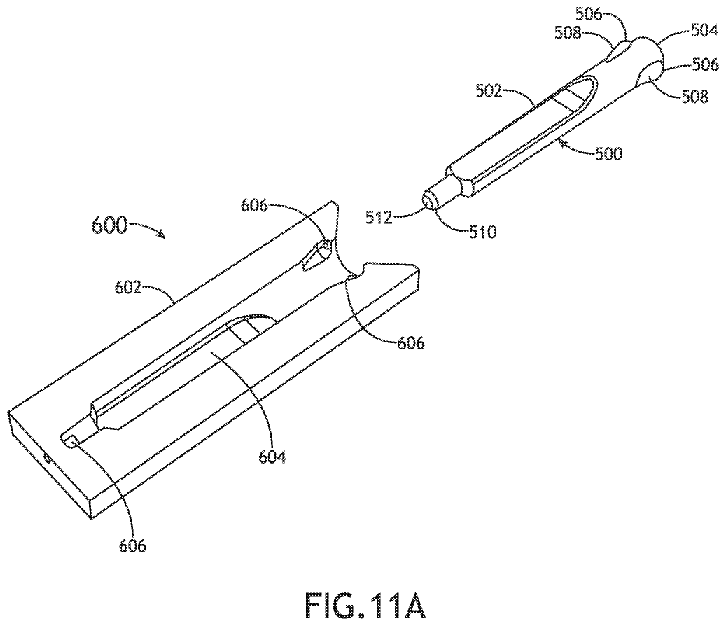

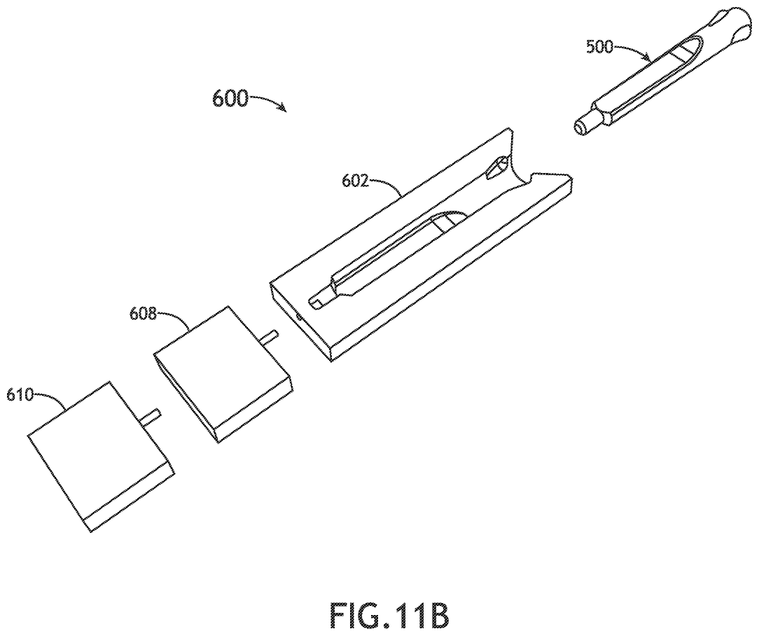

FIG. 11A is a perspective view of a system 600 that includes the OSC apparatus 500 and a docking plate 602 for the OSC apparatus 500. The docking plate 602 includes a cooperatively shaped cavity 604 that is configured to receive and hold the OSC apparatus 500. Within its cavity 604, the docking plate 602 includes plugs 606 that are configured to seal the collection ports 506 and dropper/output port 512 of the OSC apparatus 500.

The docking plate 602 may help reduce risk of infection by preventing contact with the OSC apparatus 500 and its ports after use of the OSC apparatus 500 to collect an oral specimen. For example, the docking plate 602 can allow for docking of the OSC apparatus 500 for safe transport without leaking. The docking plate 602 also provides a protective housing that can prevent infection to handlers of the OSC apparatus 500. After collecting an oral specimen, the OSC apparatus 500 is placed within the docking plate 602, which is clean. This can mitigate handling risks of highly infectious and dangerous diseases like Ebola.

In some embodiments, the docking plate 602 is part of a modular testing system/kit, as shown in FIG. 11B. Instead of expelling the specimen from the OSC apparatus 500 into a test tube, various cartridges (e.g., reagent and testing containers 608 and 610) can be connected to the docking plate 602 which may be configured to connect the cartridges to the output port 512 of the OSC apparatus 500, allowing the contents to mix. If a specific disease requires more than one reagent, you can connect them in sequential order, followed by the cartridge (testing container 610) that has an embedded test strip.

Much like the system 400 of FIG. 8, the system 600 of FIG. 11B may include the docking plate 602 as an interface to the OSC apparatus 500 and then at least one reagent container 608 and a testing container 610 that are integrated within a common structure, coupled together, or configured to be coupled together. In embodiments, the modular containers may be separated by breakable seals (e.g., pierceable barriers) that, when broken (e.g., by mating modular containers with one another), allow the specimen to flow from the OSC apparatus 500 to the one or more reagent containers 608 and then to the testing container 610. The fluid paths (e.g., openings) between the containers may be configured to provide a selected flow rate so that the specimen and reagent fluid mixture reaches the testing container 610 (optionally containing a test strip) at an appropriate flow rate and with an appropriate specimen concentration in the fluid mixture. Furthermore, to allow the reagent sufficient time to interact with the specimen, the seal separating the docking plate 602 and the reagent container 608 may be configured to be broken a selected period of time before breaking of the seal between the reagent container 608 and the testing container 610.

Although the system 600 and docking plate 602 of FIGS. 11A and 11B are described with reference to the OSC apparatus 500 illustrated in FIGS. 9A through 9C, the system 600 and docking plate 602 (with a differently cooperatively shaped cavity and appropriately located plugs) may be used with any other embodiment of the OSC apparatus described herein. For example, the system 600 and docking plate 602 can be used with OSC apparatus 100, OSC apparatus 200, OSC apparatus 700, or OSC apparatus 800 in place of OSC apparatus 500.

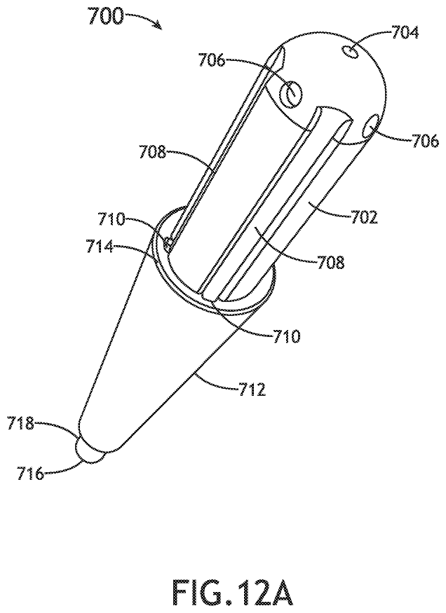

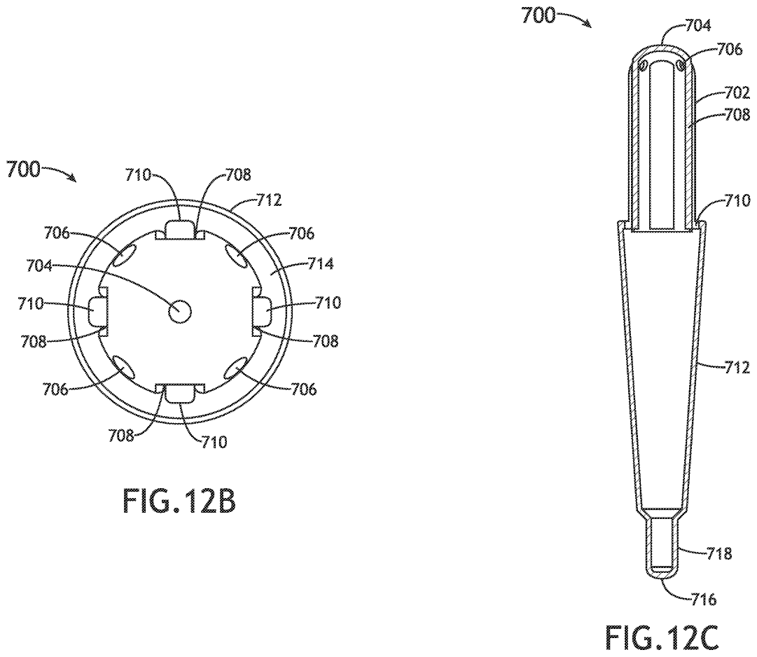

FIG. 12A through 12C illustrate another embodiment of an OSC apparatus 700, wherein the OSC apparatus 700 has separate collection and output ports disposed at opposite ends of the device, and wherein the OSC apparatus 700 further includes passive drainage features that allow the OSC apparatus 700 to capture saliva flowing across an outer surface of the OSC apparatus 700 (i.e., saliva that leaves the oral cavity without being suctioned into the collection ports). In some of the earlier embodiments, it was observed that a portion of the produced saliva would drain around and down the external walls of the device onto the users' hands. By incorporating external drain channels, any saliva that is not captured by the collection ports can be channeled down captured with passive drain ports. This prevents saliva from draining down toward the users' hands, resulting in contamination risk and loss of specimen.

In the embodiment illustrated in FIG. 12A, the OSC apparatus 700 includes a deformable container 702 with one or more collection ports 706 near its distal end 704, wherein a negative pressure is produced within the deformable container 702 when the deformable container 702 is compressed within an oral cavity, and wherein the negative pressure causes a specimen to be suctioned into the deformable container 702 through the one or more collection ports 706 as the deformable container 702 decompresses. In some embodiments, collection ports 706 are distributed about a periphery of the deformable container 702 as this configuration may be less prone to occlusion of the collection ports 706.

For passive drainage, the OSC apparatus 700 further includes one or more vertical grooves 708 formed in the outer surface of the deformable container 702. The vertical grooves 708 are configured to direct specimen fluid (e.g., saliva) flowing across the outer surface of the deformable container 702 into one or more drain ports 710 that allow the specimen to flow passively into the deformable container 702. In FIG. 12B, the OSC apparatus 700 is shown to include a plurality of collection ports 706 interleaved with a plurality of vertical grooves 708. For example, four collection ports 706 are distributed equidistantly about periphery of the deformable container 702 near its distal end 704, and the vertical grooves 708 are distributed such that every two collection ports 706 have a vertical groove 708 in between them. This arrangement is but one example of an appropriate arrangement, and in other embodiments, the OSC apparatus 700 may have a different number of collection ports 706 and vertical grooves 708 in an alternate arrangement. Furthermore, there is no requirement for the number of collection ports 706 to be the same as the number of vertical grooves 708.

In some embodiments, the deformable container 702 has a compression zone with a base structure 712 underneath. The base structure 712 may have an outermost diameter that is larger than the outermost diameter of the compression zone. This forms an outer ledge 714 that surrounds the base of the compression zone, wherein the one or more drain ports 710 are bound by the outer ledge 714, such that the outer ledge 714 and one or more drain ports 710, together, form a gutter that is configured to capture specimen fluid flowing down the one or more vertical grooves 708. In some embodiments, the compression zone has a substantially cylindrical shape while the base structure 712 is substantially conical, although other shapes may be appropriate for either portion of the deformable container 702.

As shown in FIGS. 12A and 12C, the OSC apparatus 700 also includes one or more output ports 716 for transferring the specimen from the deformable container 702 to a sample tube (e.g., a test tube, vial, collection tube, centrifuge tube, or any other type of container) for testing, transporting, and/or storing the specimen. In some embodiments, the output port 716 is at the end of a dropper tip 718 at an end of the deformable container 702 that is opposite from the collection ports 706. After collecting an oral specimen through the collection ports 706, the OSC apparatus 700 may be held with its dropper tip 718 pointed into a sample tube and squeezed to transfer its contents (i.e., the collected specimen) into the sample tube.

In some embodiments, the OSC apparatus 700 may also include a removable end cap that is configured to plug or otherwise seal the one or more collection ports 706 at the distal end 704 of the OSC apparatus 700 and/or a removable end cap that is configured to plug or otherwise seal the one or more output ports 716 at the dropper tip 718 of the OSC apparatus 700. Removable end caps can be used to seal collection and/or output ports and of the OSC apparatus 700 before and/or after specimen collection. In embodiments, the removable end caps (or any other end cap disclosed herein with regard to this or another embodiment) may comprise plugs, pressure/friction fit caps, threaded caps, or any other type of cap structure or seal.

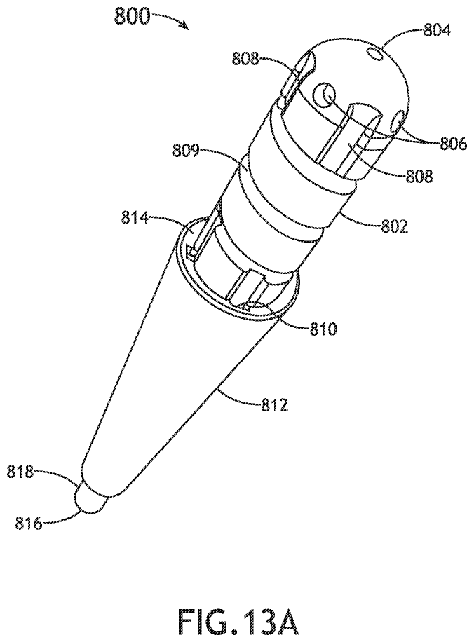

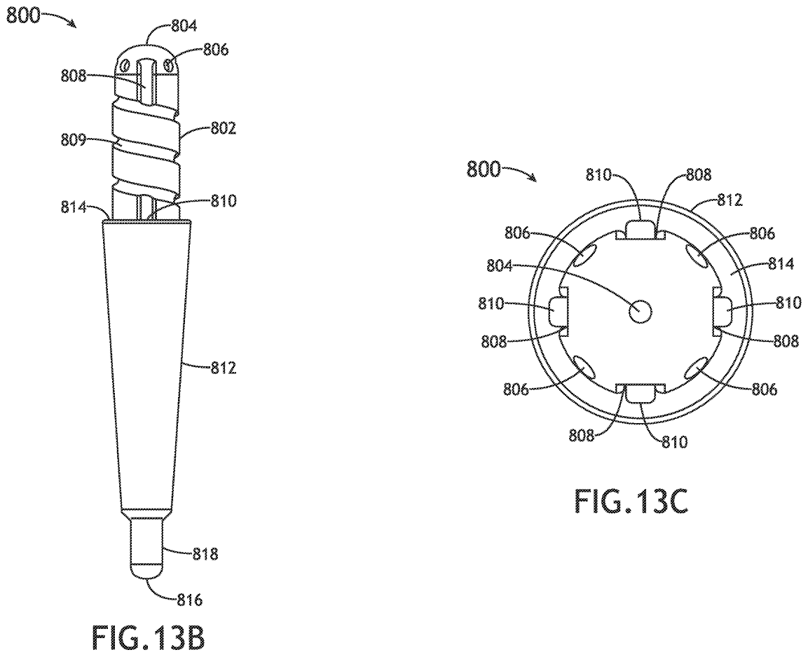

FIG. 13A through 13C illustrate another embodiment of an OSC apparatus 800, wherein the OSC apparatus 800 has separate collection and output ports disposed at opposite ends of the device, and wherein the OSC apparatus 800 further includes passive drainage features that allow the OSC apparatus 800 to capture saliva flowing across an outer surface of the OSC apparatus 800 (i.e., saliva that leaves the oral cavity without being suctioned into the collection ports).

In the embodiment illustrated in FIG. 13A, the OSC apparatus 800 includes a deformable container 802 with one or more collection ports 806 near its distal end 804, wherein a negative pressure is produced within the deformable container 802 when the deformable container 802 is compressed within an oral cavity, and wherein the negative pressure causes a specimen to be suctioned into the deformable container 802 through the one or more collection ports 806 as the deformable container 802 decompresses. In some embodiments, collection ports 806 are distributed about a periphery of the deformable container 802 as this configuration may be less prone to occlusion of the collection ports 806.

For passive drainage, the OSC apparatus 800 further includes one or more helical grooves 809 (optionally with vertical grooves 808, too) formed in the outer surface of the deformable container 802. The helical grooves 809 (or the vertical and helical grooves 808 and 809 combination) are configured to direct specimen fluid (e.g., saliva) flowing across the outer surface of the deformable container 802 into one or more drain ports 810 that allow the specimen fluid to flow passively into the deformable container 802. Embodiments with one or more helical grooves 809 may have certain advantages over embodiments with vertical grooves only (e.g., FIGS. 12A through 12C) because the one or more helical grooves 809 can capture specimen fluid flowing across any portion of the outer surface of the compression zone whereas vertical grooves are unable to capture specimen fluid that flows across non-groove portions of the outer surface of the compression zone (i.e., portions of the outer surface that are located in between two more vertical grooves). Additionally, embodiments with a combination of vertical grooves 808 and helical grooves 809 may have certain advantages because the vertical grooves 808 can help direct specimen fluid into the helical grooves 809 and can also help direct specimen fluid from the helical grooves 809 into the drain ports 810.

In some embodiments, the deformable container 802 has a compression zone with a base structure 812 underneath. The base structure 812 may have an outermost diameter that is larger than the outermost diameter of the compression zone. This forms an outer ledge 814 that surrounds the base of the compression zone, wherein the one or more drain ports 810 are bound by the outer ledge 814, such that the outer ledge 814 and one or more drain ports 810, together, form a gutter that is configured to capture specimen fluid flowing down the helical grooves 809 (or the vertical and helical grooves 808 and 809 combination). In some embodiments, the compression zone has a substantially cylindrical shape while the base structure 812 is substantially conical, although other shapes may be appropriate for either portion of the deformable container 802.

As shown in FIGS. 13A and 13B, the OSC apparatus 800 also includes one or more output ports 816 for transferring the specimen from the deformable container 802 to a sample tube (e.g., a test tube, vial, collection tube, centrifuge tube, or any other type of container) for testing, transporting, and/or storing the specimen. In some embodiments, the output port 816 is at the end of a dropper tip 818 at an end of the deformable container 802 that is opposite from the collection ports 806. After collecting an oral specimen through the collection ports 806, the OSC apparatus 800 may be held with its dropper tip 818 pointed into a sample tube and squeezed to transfer its contents (i.e., the collected specimen) into the sample tube.

In some embodiments, the OSC apparatus 800 may also include a removable end cap that is configured to plug or otherwise seal the one or more collection ports 806 at the distal end 804 of the OSC apparatus 800 and/or a removable end cap that is configured to plug or otherwise seal the one or more output ports 816 at the dropper tip 818 of the OSC apparatus 800. Removable end caps can be used to seal collection and/or output ports and of the OSC apparatus 800 before and/or after specimen collection. In embodiments, the removable end caps (or any other end cap disclosed herein with regard to this or another embodiment) may comprise plugs, pressure/friction fit caps, threaded caps, or any other type of cap structure or seal.

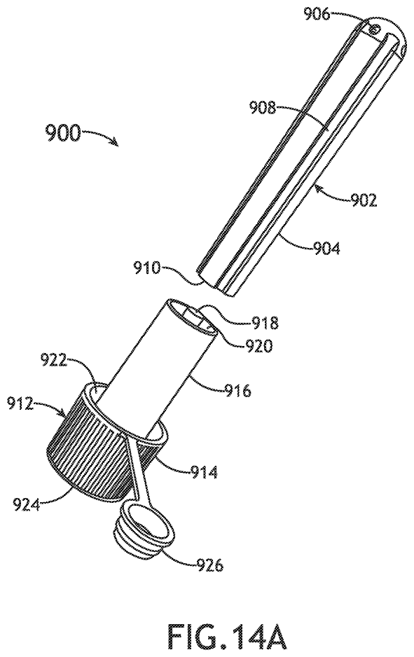

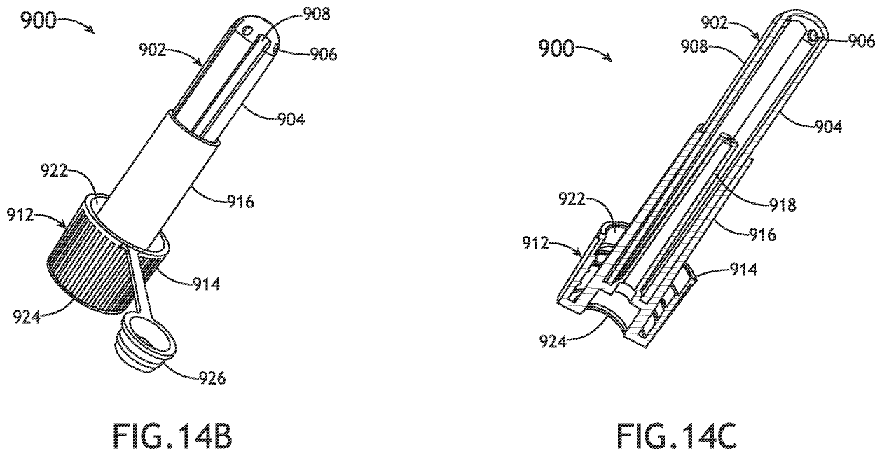

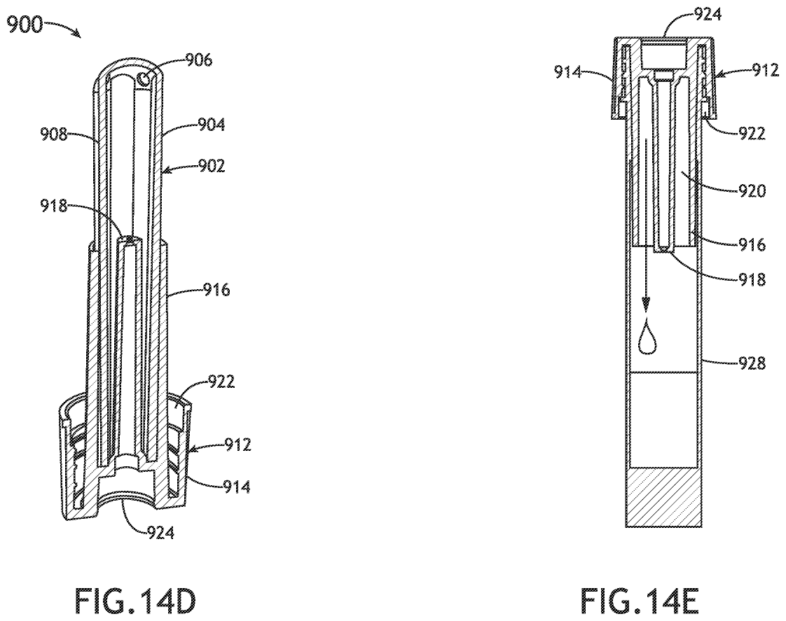

FIGS. 14A through 14E illustrate an embodiment of system 900 that includes an OSC apparatus 902 that is configured as an attachment to a base structure 912. As shown in FIG. 14A, the OSC apparatus 902 includes a deformable container 904 with one or more collection ports 906 near its distal end, wherein a negative pressure is produced within the deformable container 904 when the deformable container 904 is compressed within an oral cavity, and wherein the negative pressure causes a specimen to be suctioned into the deformable container 904 through the one or more collection ports 906 as the deformable container 904 decompresses. In some embodiments, collection ports 906 are distributed about a periphery of the deformable container 904 as this configuration may be less prone to occlusion of the collection ports 906.

For passive drainage, the OSC apparatus 902 further includes one or more vertical grooves 908 formed in the outer surface of the deformable container 904. The vertical grooves 908 are configured to direct specimen fluid (e.g., saliva) flowing across the outer surface of the deformable container 904 into a collection chamber 920 within the base structure 912. The OSC apparatus 902 may alternatively or additionally include one or more helical grooves. In some embodiments, the vertical and/or helical grooves may be arranged across the outer surface of the deformable container in a similar configuration as the vertical and/or helical grooves of OSC apparatus 700 of FIGS. 12A through 12C or OSC apparatus 800 of FIGS. 13A through 13C.

The OSC apparatus 902 is configured to be nested within the base structure 912. For example, the OSC apparatus 902 is configured to have its base inserted within the collection chamber 920 of the base structure 912, such that an outer wall 916 of the collection chamber 920 surrounds the inserted portion of the OSC apparatus 902. In some embodiments, the base structure 912 includes an inner peg 918 that fits within an opening 910 at the base of the deformable container 904 to help hold and stabilize the OSC apparatus 902 within the collection chamber 920 of the base structure 912. FIG. 14B shows the OSC apparatus 902 after it has been inserted into the base structure 912. At this point, a user can chew and/or suck on the OSC apparatus 902 to capture an oral specimen within the collection chamber 920 of the base structure 912.

As shown in FIG. 14C, in some embodiments, the peg 918 is a nozzle-like structure with a fluid path defined through the peg 918. In this case, the base structure 912 may further include a plug 926 (e.g., a tethered or fully removable plug) that is configured to seal/block an outer port 924 for the fluid path going through the peg 918. In other embodiments, the peg 918 is solid and the base structure does not include the outer port 924 or the plug 926. However, the former configuration (nozzle-like peg 918 with outer port 924 and plug 926) is advantageous as the base structure 912 can be a repurposed or dual purposed embodiment of the Nasal Specimen Collection (NSC) apparatus described in International Patent Application No. PCT/US2023/071416, which is incorporated herein by reference in its entirety. In this regard, any embodiment or feature of the NSC apparatus described in International Patent Application No. PCT/US2023/071416 may apply to the base structure 912 described herein.

As shown in FIG. 14D, the outer wall 916 of the collection chamber 920 surrounds the peg 918 so that the outer wall of the deformable container 904 can be held in between the outer wall 916 of the collection chamber 920 and the peg 918 when the OSC apparatus 902 is coupled to (e.g., nested within) the base structure 912. The base structure 912 further includes an annular shell 914 that surrounds the outer wall 916 of the collection chamber 920 with an annular cavity 922 formed in between the annular shell 914 and the collection chamber 920 of the base structure 912. The annular shell 914 is configured to interface with (e.g., mate with and/or couple to) a sample tube 928 so that contents of the collection chamber 920 (i.e., the collected oral specimen) can be emptied into the sample tube 928, as shown in FIG. 14E. For example, the annular shell 914 may include a coupling interface (e.g., cooperative threading) configured to secure the base structure 912 to an open end of the sample tube 928, wherein a distal end of the base structure 912 is pointed into the sample tube 928 so that the contents of the collection chamber 920 can be transferred (e.g., poured) into the sample tube 928. The OSC apparatus 902 is optionally removed from the base structure 912 before mating the base structure 912 with the sample tube 928 so that the OSC apparatus 902 does not encumber the transfer of specimen from the collection chamber 920 of the base structure 912 into the sample tube 928.

In embodiments, the sample tube 928 may be a specialized specimen collection and/or analysis tube. The sample tube 928 can also be any other type of fully/partially tubular container (e.g., test tube, jar, jug, beaker, etc.) or transient fluid transport device (e.g., flexible tube, pipe, hose, etc.).

The annular shell 914 (or another portion of the base structure 912) may be configured to be coupled to the sample tube 928 so that a portion of the sample tube 928 is secured within the annular cavity 922. For example, in the embodiment illustrated in FIG. 14E, the base structure 912 screws onto the sample tube 928 via threading formed on the inner surface of the annular shell 914 and complementary threading formed on an outer surface of the sample tube 928. In other embodiments, the threading may be on the outer wall 916 of the collection chamber 920 and an inner surface of the sample tube 928. The coupling interface allows the base structure 912 to seal the sample tube 928 so that the sample tube 928 can be used to safely transport fluid containing oral specimen.

The system 900 allows for an oral specimen to be collected, secured within the sample tube 928, and transported with little to no risk of contamination or potentially hazardous exposure to the handler. In some embodiments, the sample tube 928 or collection chamber 920 may also contain a specimen preservation agent and/or a testing agent so that the oral specimen is preserved or immediately tested within the sample tube 928 or collection chamber 920. For example, the sample tube 928 or collection chamber 920 may be prefilled with viral transport media (VTM) and/or an antigen detection agent.

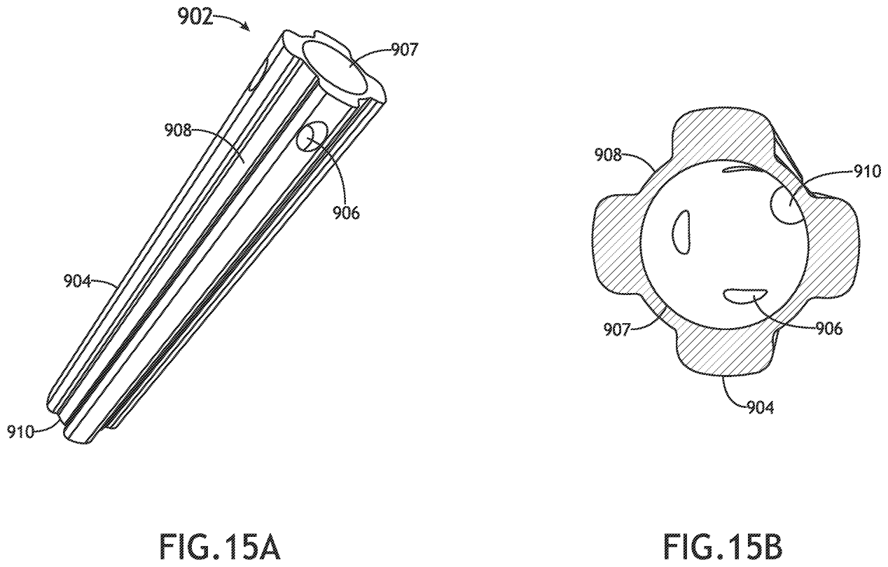

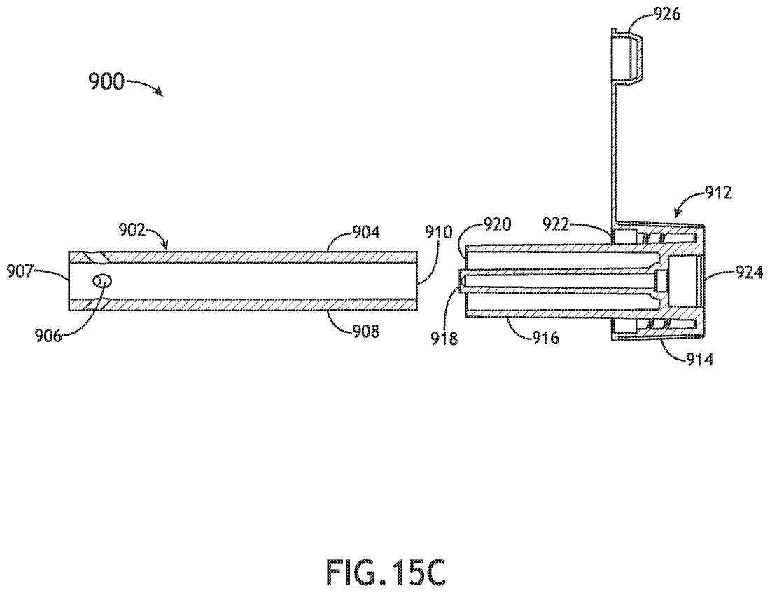

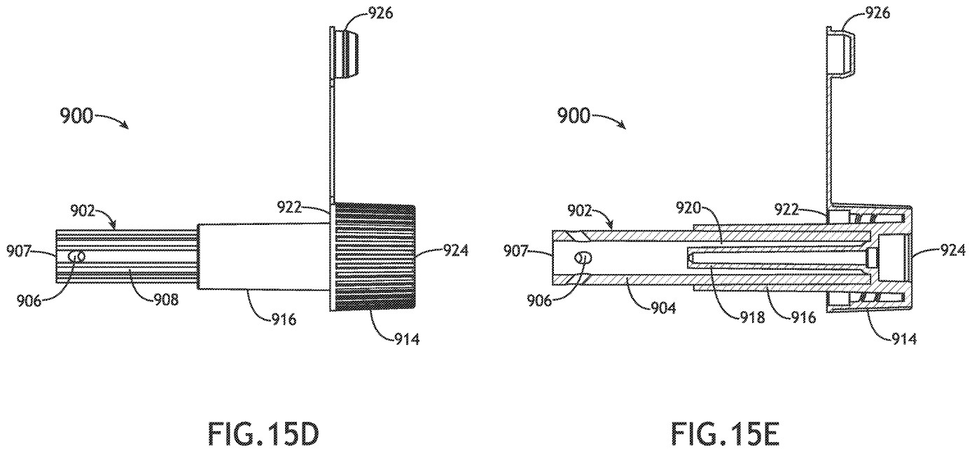

FIGS. 15A through 15E illustrate another embodiment of the OSC apparatus 902 and system 900, wherein the OSC apparatus 902 has a large central opening 907 at its distal end, making the deformable container 904 more straw-like (i.e., with large openings at each end). The OSC apparatus 902 and system 900 still function the same as the embodiment illustrated in FIGS. 14A through 14E, with an added feature-the OSC apparatus 902 in FIGS. 15A through 15E is further configured to receive passively drained specimen fluid through its central opening 907. As a user chews or sucks on the straw-like body of the deformable container, their saliva may be suctioned through the collection ports 906, flow passively down the center of the deformable container 904 (i.e., from opening 907 to 910), and/or flow down grooves 908 on the outer surface of the deformable container 904. This combination of specimen collection techniques may help ensure that a sufficient specimen is collected and can help avoid loss of saliva that falls outside the OSC apparatus 902 during the collection process.

Some alternative embodiments of the OSC apparatus may employ a syringe plunger to produce the negative pressure in the container that results in suction through the collection ports. For example, the OSC apparatus may be a syringe or have a syringe-like structure. Such embodiments may include a rigid container instead of a deformable container. However, in some embodiments, a deformable container may also be used, and the syringe plunger action can be used for suction instead of relying on compressive action, or the syringe plunger action may be used as an assistive mechanism to augment the negative pressure produced by the compressive action.

An oral specimen may comprise saliva and/or tissue samples (e.g., epithelial cells) collected from the oral cavity. In some embodiments, the deformable container may include a scraper, an abrasive surface, or a clamping mechanism at the distal end to aid in collecting tissue samples from the inside of the oral cavity (e.g., from the inner cheek). An example embodiment of a clamping mechanism may include two or more surfaces (preferably concave) that bend or invert due to compressive action (e.g., bite force, mastication, or any other application of compressive force within the oral cavity) and then pinch an inner surface of the oral cavity (e.g., inner cheek) to remove a tissue sample that can then be suctioned into the collection ports of the deformable container or captured through passive drain ports.

In some embodiments, the OSC apparatus is formed by a single mold/print. For example, the OSC apparatus may be entirely formed by a continuous 3D printed structure. Alternatively, portions of the OSC apparatus may be separately printed or cast and then assembled and/or fused together. For example, any component or portion of a component may be printed or cast separately and then assembled and/or fused together to create the final device. Separately manufacturing portions of the OSC apparatus may have advantages for injection molding processes because simpler molds and/or less material can be utilized.

Although the technology has been described with reference to the embodiments illustrated in the attached drawing figures, equivalents may be employed, and substitutions may be made herein without departing from the scope of the technology as recited in the claims. Components illustrated and described herein are examples of devices and components that may be used to implement the embodiments of the present invention and may be replaced with other devices and components without departing from the scope of the invention. Furthermore, any dimensions, degrees, and/or numerical ranges provided herein are to be understood as non-limiting examples unless otherwise specified in the claims.

1. An oral specimen collection apparatus, comprising:
a deformable container including a central opening at a distal end of the deformable container, wherein the deformable container is configured to collect, via the central opening, a specimen when the deformable container is compressed and decompressed one or more times within an oral cavity, and wherein a proximal end of the deformable container is configured to be inserted into a base structure; and
one or more grooves on an outer surface of the deformable container, wherein the one or more grooves are configured to direct fluid flowing across the outer surface of the deformable container into the base structure.

2-7. (canceled)

8. The oral specimen collection apparatus of claim 1, wherein the deformable container comprises an elongated body with a compression zone configured to be compressed within the oral cavity.

9. (canceled)

10. The oral specimen collection apparatus of claim 8, wherein the compression zone has a substantially circular, elliptical, semi-circular, or semi-elliptical shaped cross-section.

11. The oral specimen collection apparatus of claim 1, wherein the deformable container is reinforced by one or more recoil structures to assist in returning the deformable container to its original shape after the deformable container is decompressed.

12-16. (canceled)

17. The oral specimen collection apparatus of claim 1, further comprising a scraper, an abrasive surface, or a clamping mechanism at the distal end of the deformable container to aid in collecting tissue samples from within the oral cavity.

18-21. (canceled)

22. The oral specimen collection apparatus of claim 1, further comprising a flavored coating on at least a portion of the outer surface of the deformable container to promote salivation.

23-26. (canceled)

27. An oral specimen collection system, comprising:
an oral specimen collection apparatus comprising a deformable container that includes a central opening at a distal end of the deformable container, wherein deformable container is configured to collect, via the central opening, a specimen when the deformable container is compressed and decompressed one or more times within an oral cavity; and
a base structure configured to receive a proximal end of the deformable container within the base structure.

28. The oral specimen collection system of claim 27, wherein the deformable container comprises an elongated body with a compression zone configured to be compressed within the oral cavity.

29. The oral specimen collection system of claim 28, wherein the compression zone has a substantially circular, elliptical, semi-circular, or semi-elliptical shaped cross-section.

30. The oral specimen collection system of claim 27, wherein the deformable container is reinforced by one or more recoil structures to assist in returning the deformable container to its original shape after the deformable container is decompressed.

31. The oral specimen collection system of claim 27, wherein the oral specimen collection apparatus includes a scraper, an abrasive surface, or a clamping mechanism at the distal end of the deformable container to aid in collecting tissue samples from within the oral cavity.

32. The oral specimen collection system of claim 27, wherein the oral specimen collection apparatus includes one or more grooves on an outer surface of the deformable container, wherein the one or more grooves are configured to direct fluid flowing across the outer surface of the deformable container into the base structure.

33. The oral specimen collection system of claim 27, wherein the base structure is configured to be coupled to a sample tube after collecting the specimen in order to transfer the specimen to the sample tube.

34. The oral specimen collection system of claim 27, wherein the oral specimen collection apparatus includes a flavored coating on at least a portion of an outer surface of the deformable container to promote salivation.

35. An oral specimen collection apparatus, comprising:
a deformable container including a central opening at a distal end of the deformable container, wherein the deformable container is configured to collect, via the central opening, a specimen when the deformable container is compressed and decompressed one or more times within an oral cavity; and
one or more grooves on an outer surface of the deformable container, wherein the one or more grooves are configured to control a flow of fluid across the outer surface of the deformable container from the distal end of the deformable container to a proximal end of the deformable container.

36. The oral specimen collection apparatus of claim 35, wherein the deformable container comprises an elongated body with a compression zone configured to be compressed within the oral cavity.

37. The oral specimen collection apparatus of claim 36, wherein the compression zone has a substantially circular, elliptical, semi-circular, or semi-elliptical shaped cross-section.

38. The oral specimen collection apparatus of claim 35, wherein the deformable container is reinforced by one or more recoil structures to assist in returning the deformable container to its original shape after the deformable container is decompressed.

39. The oral specimen collection apparatus of claim 35, further comprising a scraper, an abrasive surface, or a clamping mechanism at the distal end of the deformable container to aid in collecting tissue samples from within the oral cavity.

40. The oral specimen collection apparatus of claim 35, further comprising a flavored coating on at least a portion of the outer surface of the deformable container to promote salivation.
